(19) United States
(12) Patent Application Publication
(10) Pub. No.: US 2009/0209873 A1
(43) Pub. Date: Aug. 20, 2009

(54) HEARTBEAT MONITORING DEVICE, SYSTEM AND METHOD

(75) Inventors: Robert Pinter, Aachen (DE); Harald Reiter, Aachen (DE); Joerg Habetha, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/373,089

(22) PCT Filed: Jul. 5, 2007
(86) PCT No.: PCT/IB07/52641

§ 371 Date: Jan. 9, 2009

(30) Foreign Application Priority Data
Jul. 14, 2006 (JP) 06117260.7

(51) Int. Cl. A61B 5/0402

(52) U.S. Cl. 600/509

(57) ABSTRACT

A heartbeat monitoring device (2) comprises: —a number of electrodes (3.1, 3.2) for sensing an ECG signal (SO) of a user (5), —a signal processing means (14) for processing a signal (S2) derived from the ECG signal and adapted to extract information related to the heartbeat of the user from the derived signal, —a standardized wireless communication module (11) for transmitting said heartbeat-related information to an external device (17). In the proposed heartbeat monitoring device the signal-processing means is implemented on a communications processor of the standardized wireless communication module.

The present invention relates to a heartbeat monitoring device, comprising a number of electrodes for sensing an electrocardiogram (ECG) signal of a user.

The present invention also relates to a method of delivering heartbeat-related information to an external device, comprising sensing an ECG signal of a user.

Furthermore, the present invention relates to a heartbeat monitoring system.

Especially during the last decade, when the discussion arose how to appropriately modify the classic healthcare systems in order to face the challenges of modern times (increasing life expectancy, drastically increased number of patients with chronic diseases requiring highly expensive treatment), the issue of a personal investment in one's own health was largely discussed. Although there is no solution in sight yet, promoting and rewarding personal investment in one's health—be it with the help of healthy nutrition, or be it by exercising regularly with a system monitoring the heart rate in the context of a fitness program—is most certainly an indispensable component of a modern and future-proof healthcare system.

State of the art commercially available heart rate monitoring systems for fitness-related applications detect the peaks in the ECG signal and send out an electromagnetic burst at every detected heartbeat in a non-standardized manner. The frequency of the burst is typically around 4 kHz. A special watch worn on the wrist receives the bursts as transmitted by a chest belt, calculates the corresponding heart rate, and displays it to the user. So the whole “intelligence” of today's chest belts for heart rate monitoring purposes is integrated in the watches. The watch is the center of the system to which the belt interfaces; the watch then interfaces to the outside world, e.g. to a PC. In other words: Chest belts for heart rate monitoring that are available today do not contain any digital signal-processing unit, e.g. a microprocessor; the signal processing is completely done with the help of analog electronics, which has the disadvantage of not delivering the best performance possible as far as the quality of heartbeat detection in the ECG is concerned. Furthermore, only dedicated hardware, e.g. a wrist watch comprising a suitable low-frequency receiver, can connect to these devices.

A number of systems are known that implement a standardized wireless link instead of the abovementioned low-frequency transmission technique. In all these cases the wireless connection is Bluetooth. However, such systems require additional hardware expenditure, i.e., if signal processing means (in the form of a microprocessor) are used for ECG peak detection then typically a further microprocessor (communications processor) is provided for Bluetooth-based signal transmission to an external device.

It is the object of the present invention to provide a heartbeat monitoring device, system and method that is improved compared to existing devices in that it achieves high-quality accurate and reliable heartbeat monitoring without having to introduce any dedicated additional hardware components.

According to a first aspect of the present invention the object is achieved by providing a heartbeat monitoring device, comprising: a number of electrodes for sensing an ECG signal of a user, a signal-processing means for processing a signal derived from the ECG signal and adapted to extract information related to the heartbeat of the user from the derived signal, and a standardized wireless communication module for transmitting said heartbeat-related information to an external device, wherein the signal-processing means is implemented on a communications processor of the standardized wireless communication module.

According to a second aspect of the present invention the object is also achieved by providing a heartbeat monitoring system comprising the heartbeat monitoring device in accordance with said first aspect of the present invention and an external device adapted to receive said heartbeat-related information from the standardized wireless communication module of the heartbeat monitoring device.

According to a third aspect of the present invention the object is further achieved by providing a method of delivering heartbeat-related information to an external device, comprising: sensing an ECG signal of a user, processing a signal derived from said ECG signal to extract therefrom heartbeat-related information of the user, transmitting the heartbeat-related information using a standardized wireless communication protocol implemented on a communications processor to the external device, wherein said processing of the derived signal is performed on the communications processor.

Thus, in accordance with a basic idea underlying the present invention, the signal-processing for peak detection in the ECG signal is implemented on the communications processor directly inside the standardized wireless communication module, e.g. a Bluetooth module. In general, any standardized wireless communication module can be employed in the context of the present invention as long as it provides sufficient (unused) processing capacity for processing said ECG signal. However, this step is far from being self-evident, since the communications processor is generally dedicated only to the processing of a corresponding standardized wireless communication protocol, e.g. the Bluetooth protocol.

Nevertheless, provided that the algorithm used for deriving heartbeat-related information does not impose an excessive load of processing power on said communications processor, it is possible to make parallel use of the communications processor for signal processing of the ECG signal or a signal derived from the ECG signal, e.g. a sampled digitized signal. The present applicant has developed heart rate determining algorithms with high-quality ECG peak detection and rather moderate processing power requirements, which as such do not form part of the present invention and which can be implemented on the communications processor of a standardized wireless communication module in accordance with the present invention.

In the context of the above-mentioned heart rate determining algorithms, the present applicant has devised an implementation of a heart rate algorithm with rather low requirements in terms of computational resources on an MSP430 processor by Texas Instruments. The algorithm requires approximately 260 KIPS (kilo instructions per second), if the ECG signal is sampled at 256 Hz, which is more than sufficient for calculating the heart rate.

If the sample rate is reduced, the computational effort scales down proportionally: For instance, if a sample rate of 100 Hz is used, which is still rather comfortable for determining the heart rate, approximately 100 KIPS of computational resources are required for the algorithm. As will be appreciated by a person skilled in the art, this represents a rather small computational load compared to the 5 . . . 10 MIPS (million instructions per second) that are required for running, e.g., the Bluetooth protocol on the communications processor.

Advantageously, embodiments of the present invention further provide a significant reduction of required board space. In order to estimate a quantitative impact of said reduction, it should be noted that in prior art implementations of ECG measuring chest belts the analog ECG amplifier section, the Bluetooth module, and the additional microcontroller (e.g. MSP430; cf. above) performing the digital signal processing of the ECG signal each require approximately one third of the total board space.

According to embodiments of the present invention, all (ECG) signal processing is implemented on the communications processor in the Bluetooth module, thus saving about 30% of board space in the present example by omitting said additional microcontroller.

Additionally, there may be a decrease in total power consumption owing to the fact that power consumption of the MSP430 microcontroller is saved. However, the beneficial effect is at least partly compensated, since power consumption of the communications processor will increase slightly if the heart rate algorithm is implemented thereon.

A more striking advantage can be expected in connection with the bill of material for practical realization of embodiments of the present invention. In the above example, cost for the MSP430 microcontroller amounts to about one fourth of the total system cost comprising said microcontroller, the Bluetooth module, and the analog amplifier. Therefore, if said microcontroller can be omitted in a specific application that requires just a calculation of the heart rate, a significant cost reduction may be achieved with the help of the present invention.

In a further embodiment of a device in accordance with the present invention the latter further comprises an analog/digital converter for sampling the ECG signal to provide a digital signal as the derived signal. In this way, digital signal processing techniques can be used for determining said heartbeat-related information, thus significantly increasing the reliability of heartbeat detection.

A corresponding embodiment of the method in accordance with the present invention comprises sampling and converting the ECG signal into a digital signal constituting the derived signal prior to said processing.

In order to facilitate usage of the device in accordance with the present invention, in another embodiment of said device, in particular for fitness-related applications, at least the electrodes are integrated in a chest belt to be worn around the chest of the user.

For further enhancing signal quality prior to deriving said signal for subsequent signal processing, in accordance with yet another embodiment of the device in accordance with the present invention the latter further comprises ECG signal amplifying and filtering means arranged between the electrodes and signal-processing means.

By employing a suitable standardized wireless communication protocol, any kind of external device using the same standardized protocol can be used for receiving said heartbeat-related information. Such external devices preferably include mobile phones, hand-held computers, PCs, or the like.

Yet another embodiment of the method in accordance with the present invention comprises extracting an indication of peaks in the ECG signal, determining a time interval between subsequent heartbeats from said indication and calculating the inverse of said time intervals. In this way, the heart rate of the user can be transmitted to/displayed on the external device.

Further advantages and characteristics of the present invention can be gathered from the following description of preferred embodiments given by way of example only with reference to the appended drawings. Features mentioned above as well as below can be used in accordance with the present invention either individually or in conjunction. The described embodiments are not to be regarded as an exhaustive enumeration but rather as examples with respect to a basic idea underlying the present invention.

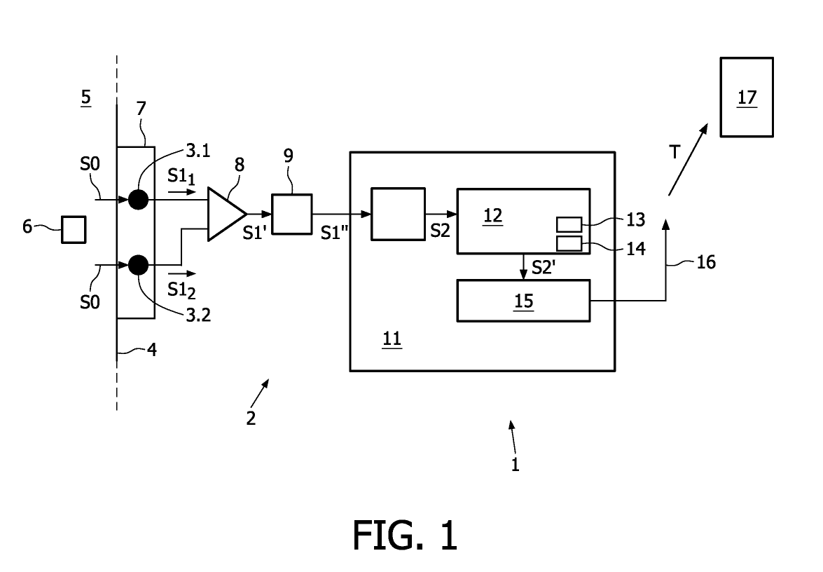

FIG. 1 is a schematic block diagram of a heartbeat monitoring device and system in accordance with the present invention; and

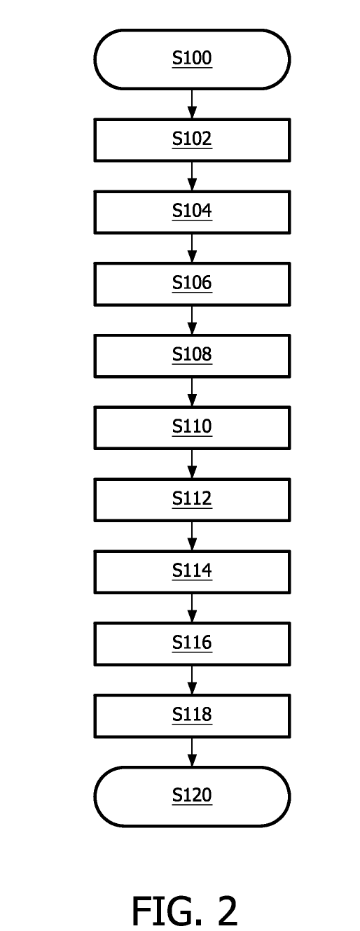

FIG. 2 is a flow chart for illustrating an embodiment of the method in accordance with the present invention.

FIG. 1 shows a schematic block diagram of a heartbeat monitoring device and system in accordance with the present invention. The heartbeat monitoring system 1 in accordance with the present invention comprises a heartbeat monitoring device 2 having a number of electrodes 3.1, 3.2 for directly contacting the skin 4 of a user 5 in the vicinity of the heart, generally depicted as box 6.

In the embodiment of FIG. 1, electrodes 3.1, 3.2 are integrated in a chest belt 7 for suitably placing said electrodes 3.1, 3.2 near the heart 6.

Electrodes 3.1, 3.2 are connected to an amplifier 8, an output of which is connected to a low-pass filter 9. Low-pass filter 9 is further connected to analog/digital converter 10 comprised in Bluetooth module 11, e.g. Bluetooth module BGB203 manufactured by the present applicant.

Besides analog/digital converter 10, Bluetooth 11 further comprises communications processor 12 implementing a Bluetooth protocol 13. Furthermore, communications processor 12 implements signal-processing means 14, a function of which will be explained in detail later.

Furthermore, Bluetooth module 11 as depicted in FIG. 1 has an RF (radio frequency) front-end 15 connected to an external antenna 16. Said antenna 16 is devised for wireless signal transmission T to an external device 17, e.g. a mobile phone, a hand-held computer, a PC, or the like.

In the embodiment of FIG. 1, elements 8 to 16 are preferably integrated into the chest belt 7 too, thus yielding a compact and easy-to-handle design of the device 2 in accordance with the present invention.

During operation of the above-described heartbeat monitoring system 1 in accordance with the present invention, electrodes 3.1, 3.2 pick up an ECG signal S0 of the heart 6 of user 5. Electrodes 3.1, 3.2 generate respective ECG signals S1₁, S1₂ (hereinafter commonly referred to as signal S1) from signal S0, which are fed to amplifier 8 for amplifying the generally small ECG signal. The amplified ECG signal S1′ is then fed to low-pass filter 9 for filtering, thus generating signal S1″ which is fed to an input of analog/digital converter 10. Analog/digital converter 10 generates sample data in the form of a digital signal S2 from the ECG signal and feeds said digital signal S2 to communications processor 12.

As stated above, communications processor 12, which is generally employed for wireless Bluetooth-based communication, implements signal processing means 14. In this way, peaks in the ECG signal indicating beats of the user's heart 6 are extracted from the sample data by applying digital signal processing implemented on communications processor 12 by means of said signal processing means 14. Using said signal processing means 14, communications processor 12 determines respective time intervals between subsequent heartbeats, calculates the inverse of said (suitably averaged) time intervals, i.e. the heart rate, and sends out corresponding heartbeat-related information in the form of signal S2′ via RF front-end 15 and external antenna 16 in accordance with the Bluetooth protocol 13, which is also implemented on communications processor 12.

In this way, the heartbeat monitoring device 2 and heartbeat monitoring system 1, respectively, in accordance with the present invention, does not require an additional micro-processor for processing the ECG signal, i.e. a signal S2 derived from the ECG signal. At the same time, the device and system in accordance with the present invention provide an alternative to known wearable heartbeat monitors which employ non-standard low-frequency transmission techniques requiring special receivers, e.g. wrist watches, instead of external device 17, which can be any device capable of Bluetooth-based communication.

In particular, since most mobile phones today comprise a Bluetooth wireless link, the present invention allows the use of a mobile phone for displaying vital signs, e.g. the heart rate, picked up by a chest belt that is equipped as described in the present document. Alternatively or additionally, a direct (galvanic) interface to a PC (not shown) is enabled/supported in the context of the present invention.

In this way, the present invention solves the problem of providing reliable heartbeat monitoring without requiring additional hardware expenditure and without relying on non-standard transmission techniques.

FIG. 2 shows a flow chart of an embodiment of the method in accordance with the present invention.

The method starts with step S100. In subsequent step S102 an ECG signal of the user is sent by means of suitably placed electrodes, as described in detail above. Then in step S104, the acquired small ECG signal is amplified, followed by a suitable low-pass filtering in step S106.

The amplified and filtered ECG signal is then transported to an analog/digital converter in step S108 for providing sampled data in the form of a digital signal.

In step S110, said digital signal is provided to digital signal processing means implemented on a communications processor for extracting therefrom information corresponding to peaks in the original ECG signal which indicate heartbeats of the user. Furthermore, in step S110 a heart rate of the user is calculated from the sequence of heartbeats.

In subsequent step S112 the calculated heartbeat-related information, i.e. the heart rate, is fed to an RF front-end for transmission in accordance with the Bluetooth protocol.

Following transmission in step S114, in subsequent step S116 said information is received by an external device that can be any device capable of receiving data via the Bluetooth protocol.

In subsequent step S118, said received data is displayed on the external device, and the method terminates with step S120.

1. A heartbeat monitoring device (2), comprising:
a number of electrodes (3.1, 3.2) for sensing an ECG signal (S0) of a user (5),
a signal-processing means (14) for processing a signal (S2) derived from the ECG signal and adapted to extract information related to the heartbeat of the user from the derived signal,
a standardized wireless communication module (11) for transmitting said heartbeat-related information to an external device (17),
wherein the signal-processing means (14) is implemented on a communications processor (12) of the standardized wireless communication module (11).

2. The device (2) of claim 1, further comprising an analogue/digital converter (10) for sampling the ECG signal (S0; S1, S1′, S1″) to provide a digital signal (S2) as the derived signal.

3. The device (2) of claim 1, characterized in that the standardized wireless communication module (11) is a Bluetooth module.

4. The device (2) of claim 1, characterized in that at least the electrodes (3.1, 3.2) are integrated in a chest belt (7) to be worn around the chest of the user (5).

5. The device (2) of claim 1, further comprising ECG signal amplifying and filtering means (8, 9) arranged between the electrodes (3.1, 3.2) and the signal processing means (14).

6. A heartbeat monitoring system (1), comprising:
the heartbeat monitoring device (2) claim 1,
an external device (17) adapted to receive said heartbeat-related information from the standardized wireless communication module (11) of the heartbeat monitoring device (2).

7. The system (1) of claim 6, characterized in that the external device (17) is a mobile phone, a hand-held computer, a PC, or the like.

8. A method of providing heartbeat-related information to an external device (17), the method comprising:
sensing an ECG signal (S0) of a user (5),
processing a signal (S2) derived from said ECG signal (S0; S1, S1′, S1″) to extract therefrom heartbeat-related information of the user (5),
transmitting the heartbeat-related information using a standardized wireless communication protocol (13) implemented on a communications processor (12) to the external device (17),
wherein said processing of the derived signal (S2) is performed on the communications processor (12).

9. The method of claim 8, further comprising sampling and converting the ECG signal (S0; S1, S1′, S1″) into a digital signal (S2) constituting the derived signal prior to said processing.

10. The method of claim 8, characterized in that processing the derived signal (S2) comprises:
extracting an indication of peaks in the ECG signal (S0; S1, S1′, S1″),
determining time intervals between subsequent heartbeats from said indication,
calculating the inverse of said time intervals.
